(19) United States
(12) Patent Application Publication
(10) Pub. No.: US 2019/0172553 A1
(43) Pub. Date: Jun. 6, 2019

(54) USING K-MERS FOR RAPID QUALITY CONTROL OF SEQUENCING DATA WITHOUT ALIGNMENT

(71) Applicant: KONINKLIJKE PHILIPS N.V., Eindhoven (NL)

(72) Inventors: Jie Wu, Cambridge, MA (US); Yee Him Cheung, Boston, MA (US)

(21) Appl. No.: 16/177,684

(22) Filed: Nov. 1, 2018

Related U.S. Application Data
(60) Provisional application No. 62/583,048, filed on Nov. 8, 2017.

(51) Int. Cl. G16B 30/00; G16B 20/00; C12Q 1/68

(52) U.S. Cl. CPC G16B 30/00; G16B 45/00; C12Q 1/68; G16B 20/00

(57) ABSTRACT

A method for evaluating nucleic acid sequencing data using a quality control analysis system, comprising: receiving a plurality of reads of a nucleic acid sequence; extracting a plurality of k-mers from the plurality of reads; identifying, using the plurality of extracted k-mers, one or more of a plurality of annotated k-mers found in the plurality of reads, wherein the plurality of extracted k-mers are stored in an annotation database, and further wherein the annotated k-mers are annotated with annotation information about the one or more nucleic acid sequences from which the annotated k-mers are generated; gathering, based on the identified annotated k-mers found in the plurality of reads, annotation information about the plurality of reads; and determining, based on the gathered annotation information, a quality control metric for at least some of the plurality of reads.

### CROSS-REFERENCE TO RELATED APPLICATION

The present application claims the benefit of U.S. Provisional Application Ser. No. 62/583,048, filed Nov. 8, 2017, which is hereby incorporated by reference in its entirety.

### FIELD OF THE INVENTION

The present disclosure is directed generally to methods and systems for quality control analysis of sequencing data.

### BACKGROUND

Next-generation sequencing (NGS) is an important tool for genomics research, and has numerous applications for discovery, diagnosis, and other methodologies. As a result, billions of NGS reads are generated all around the world every minute.

Quality control analysis of NGS data is a crucial first step required before any downstream analysis. Properly assessing the quality of the NGS data prior to subsequent analysis can reduce or prevent misinterpretation of data, misdiagnosis, and other undesirable downstream effects.

There are established metrics for quality control analysis of NGS data. Typically, these analyses are performed before or after aligning the reads to the genome. Tools like FASTQC, for example, are used to review raw sequencing data such as fastq files, and typically report information such as base qualities, GC content, sequence duplication levels, and potential adapter contents. These metrics can be derived without knowing the locations of the reads relative to the nucleic acid being sequenced. There are advanced quality control metrics which cannot be extracted directly from read sequences, such as mapping rates, rRNA read percentages, read distributions (including exonic, intronic, intergenic, and others), 5′/3′ bias, and numbers of detected transcripts. Accordingly, it is necessary for reads to be aligned with the genomic reference, and the aligned reads are then analyzed by programs such as RNA-SeQC to extract these quality control metrics.

However, aligning the obtained reads to the genomic reference is a time-consuming process that can take hours. As a result, these advanced quality control metrics can only be obtained long after the reads have been generated. This significant time delay between generating reads and obtaining quality control information prevents corrective measures and can result in inefficiency and added cost.

### SUMMARY OF THE INVENTION

There is a continued need for real-time or near real-time quality control analysis of sequencing data.

The present disclosure is directed to inventive methods and systems for quality control analysis of sequencing data without alignment to a genomic reference. Various embodiments and implementations herein are directed to a system in which a plurality of k-mers from one or more genomic references are annotated in a database with information about the k-mer, such as species, location, and other possible information. K-mers are extracted from reads generated during sequencing, and these extracted k-mers are used to find matching annotated k-mers in a quality control analysis. An annotation associated with a k-mer in the database matching an extracted k-mers provides information about the species of the read, the most likely origination of the read, and/or other information about the read. Annotation information can be collated and summarized to provide one or more quality control metrics for an entire dataset.

Generally in one aspect, a method for evaluating nucleic acid sequencing data using a quality control analysis system is provided. The method includes: (i) receiving, by the quality control analysis system, a plurality of reads of a nucleic acid sequence; (ii) extracting, by a processor of the quality control analysis system, a plurality of k-mers from the plurality of reads; (iii) identifying, using the plurality of extracted k-mers, one or more of a plurality of annotated k-mers found in the plurality of reads, wherein the plurality of annotated k-mers are generated from one or more nucleic acid sequences and stored in an annotation database of the quality control analysis system, and further wherein the annotated k-mers are annotated with annotation information about the one or more nucleic acid sequences from which the annotated k-mers are generated; (iv) gathering, based on the identified annotated k-mers found in the plurality of reads, annotation information about the plurality of reads; and (v) determining, based on the gathered annotation information, a quality control metric for at least some of the plurality of reads.

According to an embodiment, the method further includes the step of reporting the determined quality control metric.

According to an embodiment, the method further includes the step of responding to the determined quality control metric.

According to an embodiment, the method further includes the steps of: (i) generating, from a nucleic acid sequence, the plurality of annotated k-mers; (ii) storing the generated plurality of k-mers in the annotation database; and (iii) annotating one or more of the stored plurality of k-mers with annotation information.

According to an embodiment, the annotation information comprises information about a location of the annotated k-mer within the nucleic acid sequence. According to an embodiment, the annotation information comprises information about a characteristic of the nucleic acid sequence from which the annotated k-mer was generated.

According to an embodiment, the quality control metric is a measurement of contamination of the reads, depth or coverage of the plurality of reads, and/or an identification of one or more species from which the plurality of reads were generated.

According to an embodiment, the quality control metric is determined prior to alignment of the received reads to a genomic sequence.

According to an aspect is a system configured to evaluate nucleic acid sequencing data. The system includes: an annotation database comprising a plurality of annotated k-mers generated from one or more nucleic acid sequences, wherein the plurality of annotated k-mers are annotated with annotation information about the one or more nucleic acid sequences from which the annotated k-mers are generated; and a processor comprising a k-mer extractor configured to extract a plurality of k-mers from a plurality of reads of a nucleic acid sequence, and a k-mer analyzer configured to: (i) identify, using the plurality of extracted k-mers, one or more of the plurality of annotated k-mers in the annotation database found in the plurality of reads; (ii) gather, based on the identified annotated k-mers found in the plurality of reads, annotation information about the plurality of reads; and (iii) determine, based on the gathered annotation information, a quality control metric for at least some of the plurality of reads.

According to an embodiment, the system further includes an annotator configured to: (i) generate the plurality of annotated k-mers from the one or more nucleic acid sequences; (ii) store the generated plurality of k-mers in the annotation database; and (iii) annotate one or more of the stored plurality of k-mers with annotation information.

According to an embodiment, the processor is configured to respond to the determined quality control metric.

According to an embodiment, the system further includes a user interface configured to provide the determined quality control metric to a user.

According to an embodiment, the quality control metric is determined prior to alignment of the received reads to a genomic sequence.

According to an embodiment, the k-mer extractor is configured to extract k-mers from the plurality of reads using a sliding window method.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for quality control analysis of sequencing data using a quality control analysis system, in accordance with an embodiment.

FIG. 2 is a flowchart of a method for quality control analysis of sequencing data using a quality control analysis system, in accordance with an embodiment.

FIG. 3 is a schematic representation of a quality control analysis system, in accordance with an embodiment.

### DETAILED DESCRIPTION OF EMBODIMENTS

The present disclosure describes various embodiments of a system and method for quality control analysis of sequencing data. More generally, Applicant has recognized and appreciated that it would be beneficial to provide a system that provides quality control analysis of sequencing data without requiring alignment of the reads to the genomic reference. The system comprises a database of k-mers extracted from one or more genomic references and annotated with information about the k-mer, such as species, location, and/or other information. As NGS data is obtained, k-mers are extracted from the reads and are used to identify one or more annotated k-mers in the annotated k-mer database. The annotation information associated with each identified k-mer in the database provides data about the species of the read, the most likely origination of the read, and/or other information about the read. Annotation information can be collated and summarized to provide one or more quality control metrics for an entire dataset.

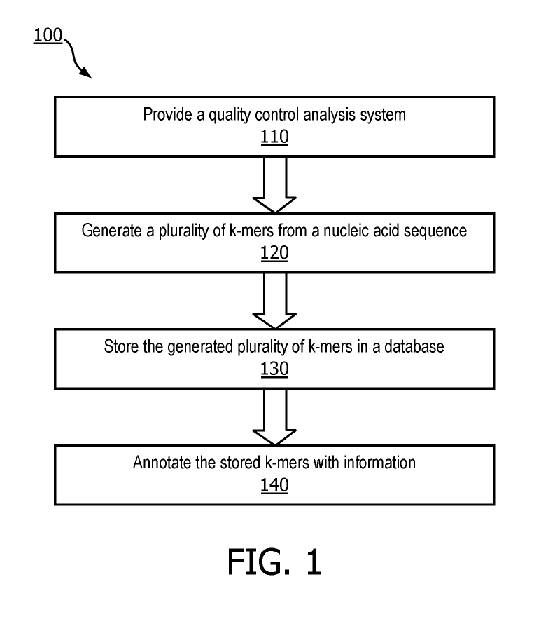

Referring to FIG. 1, in one embodiment, is a flowchart of a method 100 for quality control analysis of sequencing data. At step 110 of the method a quality control analysis system is provided. The quality control analysis system may be any of the systems described or otherwise envisioned herein.

At step 120 of the method, a plurality of k-mers are generated from one or more nucleic acid sequences. The nucleic acid sequence can be any sequence, such as a genomic sequence. According to an embodiment, k-mer generation is performed by an automated algorithm that receives a nucleic acid sequence and generates a plurality of k-mers from that sequence. Input into the algorithm may include the nucleic acid sequence, the desired k-mer length or lengths, and information about a location to store the generated k-mers, among many other possible variables.

K-mers may be generated from pre-determined target regions of the genomic sequence of an organism, or may be generated from all or portions of the genomic sequence of the organism. According to an embodiment, one or more regions of the genomic sequence may be targeted for k-mer generation based on a desired use or output of the quality control system. As just one example, if only certain regions of a sequencing output will undergo downstream analysis, only these certain regions may be targeted for quality control and thus the quality control system may be instructed or designed to generate k-mers only for these certain regions. Alternatively, the entire genomic sequence of an organism may be utilized to generate k-mers.

K-mers may be generated from a single organism or species, or may be generated from a plurality of organisms or plurality of species. For example, k-mers from the genomic sequences of common, possible, or known contaminating organisms may be generated in order to detect these contaminating organisms during the quality control analysis. Additionally, k-mers from the genomic sequences of different organisms that could be found in complex samples can be generated in order to detect and analyze sequence data from multiple different organisms during the quality control analysis.

The k-mers extracted from the one or more nucleic acid sequences can be the same length or can be a variety of different lengths. As just one example, the generated k-mers may be approximately 14 bases, although longer and shorter k-mers are possible. A large K may reduce the probability of finding multiple hits in the reference genome, which may result in multiple types of annotations of the K-mer. For example, a smaller K may be mapped to many places in the genome and does not provide useful unique annotation information. However, a larger K will also increase the amount of memory required for storing the generated k-mers. A shorter K may decrease the amount of memory required for storing the generated k-mers. Accordingly, determining an optimal K may depend on a variety of factors such as computing power and memory size, among other factors.

At step 130 of the method, the plurality of generated k-mers are stored in a database, such as a database of the quality control analysis system. Alternatively, the generated k-mers may be stored in a remote database and accessed remotely by the quality control analysis system. The generated k-mers may be temporarily stored in a first database for annotation as described in a subsequent step, prior to being permanently stored in a second database.

At step 140 of the method, one or more of the stored plurality of k-mers are annotated with information. A piece of information is associated in memory with a particular k-mer such that retrieving or identifying the particular k-mer also retrieves or identifies the associated piece of information. The annotation can be any information about the k-mer, about the genome or genomic sequence from which the k-mer was generated, about the organism from which the k-mer was obtained, about any genotype or phenotype of the organism, and/or any other piece of information.

According to an embodiment, the generated k-mers are annotated with information about a characteristic of the nucleic acid sequence from which the annotated k-mer was generated, such as location information and/or gene structure information. For example, the generated k-mers may be annotated with one or more of the following annotations, among many other possible annotations:
EXON—the k-mer is found in an annotated exon;
CDS—the k-mer is found in an annotated coding region of a gene;
3UTR/5UTR—the k-mer is found in an annotated 3UTR/5UTR part of a gene;
INTRON—the k-mer is found in an annotated intron;
GENE—the k-mer is found in an annotated gene;
PROMOTER—the k-mer is found in the promoter region of an annotated gene;
UPSTREAM or DOWNSTREAM—the k-mer is found in flanking region (such as within 2,000 nucleotides) of an annotated gene; and/or
INTERGENIC—the k-mer is found in a region far away (such as at least 2,000 nucleotides) from any annotated genes.

According to an embodiment, the generated k-mers are annotated with information related to the source of the read. For example, the generated k-mers may be annotated with one or more of the following annotations, among many other possible annotations:
rRNA—the k-mer is found in an rRNA sequence;
mtDNA—the k-mer is found in a mitochondria DNA sequence;
REPETITIVE—the k-mer is found in a repetitive region;
SPECIES—the species the K-mer is from; and/or
ADAPTOR or PRIMER—the k-mer is a part of a sequencing adapter or primer.

Other possible annotations include, but are not limited to:
STRAND—the strand from which the k-mer is derived;
DISTANCE—the distance to 3′ site of transcript;
GENE ID—to estimate expression of a gene;
GENE TYPE—a classification or other identifier for the gene from which the k-mer is derived; and/or
RELATED PATHWAY—a pathway classification or identifier for the region or gene from which the k-mer is derived.

Although the above annotations are provided with a specific label such as “EXON” or “STRAND,” it should be recognized that any identifier can be utilized to indicate the label.

Referring to TABLE 1 is an example of a database table of annotated k-mers. The database table comprises a set of data elements (k-mer values and the corresponding annotation) in vertical columns and horizontal rows. Each row comprises a stored k-mer and an associated annotation. For example, row number 1 comprises a k-mer having the value “GCTAGCCGTGAACGTCGATG,” which is annotated as being from an intron (“INTRON”) of a particular gene or multiple genes (“[GENE NAME]”). The database may be queried and maintained using any method, including but not limited to SQL.

TABLE 1
Sample database table of annotated k-mers.

| Row | | |
|---|---|---|
| Number | Stored k-mer | Annotation |
| 1 | CTGTTGAGACGGTC | INTRON: [GENE NAME] |
| 2 | GCGGCGGATACAGT | [GENE NAME]: 3UTR: |
| | | CDS: EXON |
| 3 | ATTAGTGTTGCGGG | INTERGENIC |
| 4 | TGTACGACCGAGAT | EXON: 5UTR: [GENE NAME] |
| 5 | ACGCAGCCGCAGAA | INTERGENIC: [ORGANISM] |
| 6 | CGTAGAGGCCTTCA | CDS: EXON: [SPECIES] |

According to an embodiment, k-mers with ambiguous and/or conflicting origination data or other conflicting data may be removed by the quality control analysis system. For example, if a k-mer can be found in both an exon and intron, both originations can be removed. In the end, only useful K-mers and their useful annotations are stored in the query table. Note that one K-mer can have multiple features/originations assigned, as long as the properties do not conflict with each other. Tests need to be done to determine the most optimal K.

According to an embodiment, the quality control analysis system comprises a user interface, algorithm, and/or other element that enables manual and/or automated annotation of the generated k-mers. For example, a user or programmer may provide a rule set that is utilized for annotation. One such rule set may indicate that k-mers located within an exon region are automatically annotated with the label “EXON,” and that k-mers located within an intron region are automatically annotated with the label “INTRON.” This location information can be entered manually, although such an annotation process would be time intensive. Typically, location information or other annotation information can be retrieved from a database such as a database of location information. Many publicly-available and private databases comprise information about genomic sequences that can be utilized by rule sets and annotation algorithms to annotate the generated k-mers.

With a completed database of generated k-mers that are adequately annotated to allow for quality control analysis by the system, the database can be queried using reads generated from a nucleic acid sequence.

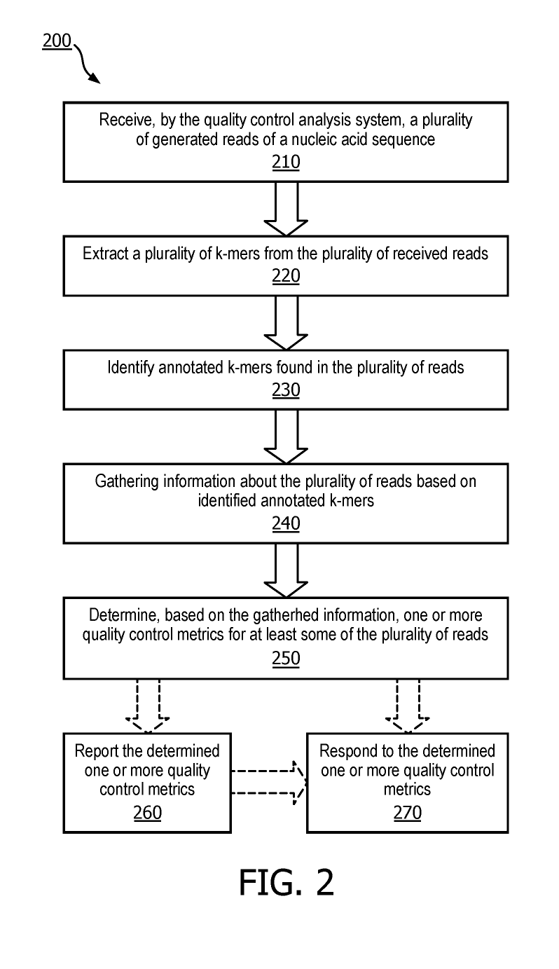

Referring to FIG. 2 is a flowchart of a method 200 for quality control analysis of sequencing data using a quality control analysis system, which may be any of the systems described or otherwise envisioned herein.

At step 210 of method 200, a plurality of reads of a nucleic acid sequences are generated, and are received by the quality control analysis system. The reads can be generated using any sequencing method, device, or system, including but not limited to high-throughput sequencers. The reads may be generated from a known or an unknown sample, which may contain DNA or RNA from one or multiple organisms. The reads can be stored for analysis. For example, the generated reads may be stored in any format, including but not limited to the FASTQ and related formats.

The reads can be provided to the quality control analysis system immediately upon generation, or can be provided to the quality control analysis system after a sequencing project is completed. Accordingly, the quality control analysis system may be in wired and/or wireless communication with a sequencer generating reads. Alternatively, the quality control analysis system may receive reads from any other manual or automated method or process.

At step 220 of the method, the quality control analysis system extracts k-mers from the received plurality of reads. Accordingly, the quality control analysis system comprises an algorithm such as a script that is programmed or configured to extract one or more k-mers from a read, typically based on one or more predetermined rules. For example, one of the predetermined rules may be a k-mer length that defines the length of each extracted k-mer. According to an embodiment, a read comprising N nucleotides can be split into maximum N−k+1 different k-mers.

According to an embodiment, the k-mers are extracted using a sliding window methodology. For example, a read may be 50 nucleotides while the algorithm is configured to extract k-mers of 20 nucleotides. The extraction algorithm may be configured to randomly sample the read with one or more generated k-mers, where the sample size may be determined by the rule set and/or based on the length of the read. According to an embodiment, the k-mers can be repeatedly sampled until an origination for a read can be determined.

The extraction algorithm may be configured to extract k-mers using a sliding window, where the parameters are pre-programmed or derived from a rule set that may be user-defined. For example, the extraction algorithm may generate a plurality of different k-mers from a 100 nucleotide read, where the k-mers may be overlapping and/or non-overlapping.

At step 230 of the method, the quality control analysis system identifies annotated k-mers in the annotation database found in the extracted k-mers. According to one embodiment, this can be accomplished by comparing each k-mer extracted from a sequencing read to each stored k-mer in each row of the annotation database and identifying when there is a match between the extracted and stored k-mers. According to another embodiment, this can be accomplished without row by row comparison, such as by extracting k-mer information from a hash table. For example, extracted k-mers may be mapped to annotated k-mers in the hash table using a hash function. Other methods of identifying annotated k-mers in the annotation database found in the extracted k-mers are also possible.

The system may require an exact match between the extracted and stored k-mers, or may be programmed or otherwise configured to allow a certain level of mismatch to occur between the extracted and stored k-mer while still identifying them as a match. According to some embodiments, some quality control analyses may have more room for mismatch and approximation or estimation of data quality. Accordingly, the quality control analysis system may comprise a rule set and/or a user interface that may be utilized to set an optional mismatch level.

At step 240 of the method, the quality control analysis system receives or gathers information about the plurality of reads based on the identified annotated k-mers in the annotation database that have a match in the extracted k-mer. When an extracted k-mer is identified in step 230 as matching an annotated k-mer in the annotation database, the annotation information associated with that matching annotated k-mer can be read, copied, moved, or otherwise received or gathered. For example, the annotation information may be copied to a new table in the annotation database, and/or to a new table in a different database. Many other methods for receiving or gathering the identified annotation information are possible.

At step 250 of the method, the quality control analysis system determines, based on the received and/or gathered annotation information, one or more quality control metrics for at least some of the plurality of reads. The identified annotation information can be collated or analyzed using any method or system to extract a sequencing quality control metric or parameter.

According to an embodiment, the identified annotation information is analyzed to determine how the reads are distributed as to exonic vs. intronic, UTR vs. CDS, gene vs. intergenic, specific locations of the genome, and/or many other possible distributions. According to an embodiment, the identified annotation information is analyzed to determine how the reads are distributed along the transcripts in order to evaluate 3′ to 5′ bias. According to yet another embodiment, the identified annotation information is analyzed to determine whether there is contamination from rRNA, mtDNA, and/or other species or organisms, among other possible contaminations. Accordingly, the identified annotation information is analyzed to determine a quantitative or qualitative measurement of contamination of the reads. According to another embodiment, the identified annotation information is analyzed to determine strand specificity.

At optional step 260 of the method, the quality control analysis system reports the determined one or more quality control metrics. Accordingly, the quality control analysis system may comprise a user interface configured to provide real-time and/or cumulative quality control metrics to a user. The system may also be programmed or configured to generate an electronic and/or printed report of the one or more quality control metrics. The report may include information about identified k-mers, annotation information, and/or many other types of information.

At optional step 270 of the method, the quality control analysis system may respond to the determined one or more quality control metrics. The response may be automatic based on a rule set, and/or may be based on user input. The system may determine, based on the generated quality control metric, the utility of the read and whether to proceed with sequence alignment or other processing. For example, if a quality control metric indicates that the analyzed reads have a high level of contamination, the system or the user may determine that a better sample is required for sequencing and that alignment and other downstream processing should be prevented or abandoned. As another example, if a quality control metric indicates that the analyzed reads suggest a sequencing coverage or depth that is unacceptable or below a predetermined threshold, the system or the user may determine that a better sample is required for sequencing and that alignment and other downstream processing should be prevented or abandoned. Similarly, if the quality control metric indicates that the analyzed reads suggest a sequencing coverage or depth that is acceptable or above a predetermined threshold, the system or the user may send the reads for alignment and/or other downstream processing.

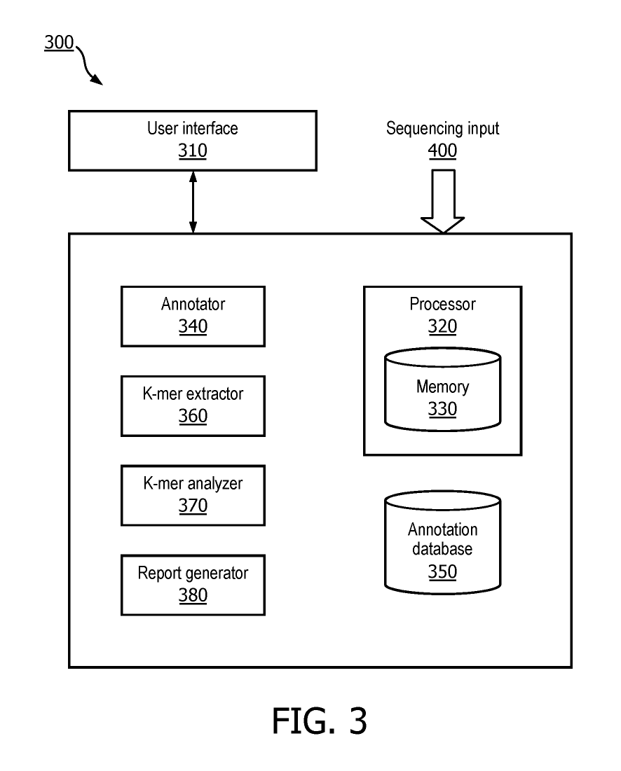

Referring to FIG. 3, in one embodiment, is a schematic representation of a quality control analysis system 300. System 300 can comprise any of the modules, elements, databases, processors, and/or other components described or otherwise envisioned herein.

According to an embodiment, system 300 comprises a user interface 310 to receive input from a user, and/or to provide information such as a determined quality control metric to a user. For example, the user interface may be used to generate or modify a rule set utilized by the system for the quality control analysis. The user interface can be any device or system that allows information to be conveyed and/or received, such as a speaker or screen, among many other types of user interfaces. The information may also be conveyed to and/or received from a computing device or an automated system. The user interface may be located with one or more other components of the system, or may located remote from the system and in communication via a wired and/or wireless communications network.

According to an embodiment, system 300 comprises a processor 320 which performs one or more steps of the method, and may comprise one or more of the modules. Processor 320 may be formed of one or multiple modules, and can comprise, for example, a memory 330. Processor 320 may take any suitable form, including but not limited to a microcontroller, multiple microcontrollers, circuitry, a single processor, or plural processors. Memory 330 can take any suitable form, including a non-volatile memory and/or RAM. The non-volatile memory may include read only memory (ROM), a hard disk drive (HDD), or a solid state drive (SSD). The memory can store, among other things, an operating system. The RAM is used by the processor for the temporary storage of data. According to an embodiment, an operating system may contain code which, when executed by the processor, controls operation of one or more components of system 300.

According to an embodiment, system 300 comprises an annotator 340, which may be a processor, a component of one or more processors, and/or a software algorithm. Annotator 340 generates a plurality of k-mers from a nucleic acid sequence, stores the generated plurality of k-mers in an annotation database 350, and annotates one or more of the stored plurality of k-mers with annotation information. Annotation database 350 may be a component of the system or may be stored locally or remotely and in periodic and/or continuous communication with the system. The database may for example comprise one or more data tables such as the data table shown in Table 1.

The annotator 340 may receive input from a rule set and/or a user that guides or otherwise provides parameters for the generation of k-mers. Input may include the nucleic acid sequence, the desired k-mer length or lengths, and information about a location to store the generated k-mers, among many other possible variables. K-mers may be generated using any known method, including but not limited the methods described or otherwise envisioned herein.

The annotator 340 stores the generated k-mers in memory with annotation information such that retrieving or identifying a particular stored k-mer also retrieves or identifies the associated piece of information. The annotation can be any information about the k-mer, about the genome or genomic sequence from which the k-mer was generated, about the organism from which the k-mer was obtained, about any genotype or phenotype of the organism, and/or any other piece of information. According to an embodiment, a user may manually annotate—or provide annotation parameters for—the generated k-mers. Once the annotation database 350 is populated with k-mers and associated annotation information, the database can be queried using reads generated from a nucleic acid sequence.

According to an embodiment, system 300 comprises a k-mer extractor 360 which may be a processor, a component of one or more processors, and/or a software algorithm. System 300 receives sequencing input 400, comprising a plurality of sequencing reads, from a connected or remote sequencer or other sequence data source. K-mer extractor 360 is configured to extract k-mers from these received reads using any method, including but not limited to the methods described or otherwise envisioned herein. For example, the k-mer extractor 360 may be programmed or configured to extract one or more k-mers from a read, typically based on one or more predetermined rules. For example, one of the predetermined rules may be a k-mer length that defines the length of each extracted k-mer. K-mer extractor 360 may be configured to extract k-mers using a sliding window, where the parameters are pre-programmed or derived from a rule set that may be user-defined. For example, k-mer extractor 360 may generate a plurality of different k-mers from a 100 nucleotide read, where the k-mers may be overlapping and/or non-overlapping.

According to an embodiment, system 300 comprises a k-mer analyzer 370 which may be a processor, a component of one or more processors, and/or a software algorithm. K-mer analyzer 370 is configured to identify annotated k-mers stored in the annotation database 350 that are found in the extracted k-mers. The k-mer analyzer identifies annotated k-mers in the annotation database that have a match in the extracted k-mer by, for example, comparing each k-mer extracted from a sequencing read to each stored k-mer in each row of the annotation database, or using a hash table or other analysis. When there is a match between an extracted k-mer and a stored k-mer, the annotation information associated with that matching annotated k-mer can be read, copied, moved, or otherwise received or gathered. For example, the annotation information may be copied to a new table in the annotation database, and/or to a new table in a different database. As another example, annotation information may be gathered for any annotated k-mer to which an extracted k-mer has been mapped in the hash table. Many other methods for receiving or gathering the identified annotation information are possible.

According to an embodiment, the k-mer analyzer 370 may require an exact match between the extracted and stored k-mers, or may be programmed or otherwise configured to allow a certain level of mismatch to occur between the extracted and stored k-mer while still identifying them as a match.

The k-mer analyzer 370 determines, based on the gathered annotation information, one or more quality control metrics for the received reads. The k-mer analyzer can determine any quality control metric based on the gathered annotation information. For example, the k-mer analyzer can analyze the identified annotation information to determine how the reads are distributed as to exonic vs. intronic, UTR vs. CDS, gene vs. intergenic, specific locations of the genome, and/or many other possible distributions. According to an embodiment, the k-mer analyzer can analyze the identified annotation information to determine how the reads are distributed along the transcripts in order to evaluate 3′ to 5′ bias. According to yet another embodiment, the identified annotation information is analyzed to determine whether there is contamination from rRNA, mtDNA, and/or other species or organisms, among other possible contaminations. Many other quality control metrics are possible.

According to an embodiment, system 300 comprises a report generator 380 which may be a processor, a component of one or more processors, and/or a software algorithm. Report generator 380 is configured to generate a report of the one or more determined quality control metrics. The report may be provided to a user via user interface 310. System 300 may also be configured to generate a printed report of the one or more quality control metrics. A report may include information about identified k-mers, annotation information, and/or many other types of information.

System 300 may be further configured to make one or more decisions in response to the determined one or more quality control metrics. The response may be automatic based on a rule set, and/or may be based on user input. For example, the system may be configured or programmed to determine, based on the generated quality control metric, the utility of the read and whether to proceed with sequence alignment or other processing, among many other possible decisions or determinations.

According to an embodiment, the method for evaluating nucleic acid sequencing data significantly improves the functionality, speed, and efficiency of a quality control analysis device or system. The quality control analysis device or system requires significantly less processing power and time to achieve the same or an improved result of quality control analysis of sequencing data, compared to traditional systems. For example, existing quality control methods and systems require alignment of the reads to the reference sequence before quality can be assessed, which can require several hours. In contrast, the improved quality control methods and systems described herein can be performed or used in real-time as reads are generated, and/or can be performed in as little as 5-10 minutes using a generated dataset. Accordingly, quality control metrics are generated in minutes rather than hours. Not only does this improve the speed of the system, it also improves efficiency. Quality control systems require massive amounts of computing power to perform alignment followed by quality control. Using the systems and methods described herein, the quality control system performs quality control without alignment, which significantly reduces computing needs. Furthermore, when the system determines that sequencing data is problematic or potentially problematic based on a quality control metric, the system can terminate the sequencing in real-time. This saves significant computing power and improves efficiency. For example, if the system determines that the sequencing data is contaminated with sequences from a contaminating species, the system can terminate the sequencing. Not only is processor efficiency increased when sequencing is terminated due to quality control data, but sequencing reagents are saved, further reducing inefficiency.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for evaluating nucleic acid sequencing data using a quality control analysis system, comprising:
receiving, by the quality control analysis system, a plurality of reads of a nucleic acid sequence;
extracting, by a processor of the quality control analysis system, a plurality of k-mers from the plurality of reads;
identifying, using the plurality of extracted k-mers, one or more of a plurality of annotated k-mers found in the plurality of reads, wherein the plurality of annotated k-mers are generated from one or more nucleic acid sequences and stored in an annotation database of the quality control analysis system, and further wherein the annotated k-mers are annotated with annotation information about the one or more nucleic acid sequences from which the annotated k-mers are generated;
gathering, based on the identified annotated k-mers found in the plurality of reads, annotation information about the plurality of reads; and
determining, based on the gathered annotation information, a quality control metric for at least some of the plurality of reads.

2. The method of claim 1, further comprising the step of reporting the determined quality control metric.

3. The method of claim 1, further comprising the step of responding to the determined quality control metric.

4. The method of claim 1, further comprising the steps of:
generating, from a nucleic acid sequence, the plurality of annotated k-mers;
storing the generated plurality of k-mers in the annotation database; and
annotating one or more of the stored plurality of k-mers with annotation information.

5. The method of claim 4, wherein the annotation information comprises information about a location of the annotated k-mer within the nucleic acid sequence.

6. The method of claim 4, wherein the annotation information comprises information about a characteristic of the nucleic acid sequence from which the annotated k-mer was generated.

7. The method of claim 1, wherein the quality control metric is a measurement of contamination of the reads, depth or coverage of the plurality of reads, and/or an identification of one or more species from which the plurality of reads were generated.

8. The method of claim 1, wherein the quality control metric is determined prior to alignment of the received reads to a genomic sequence.

9. A system configured to evaluate nucleic acid sequencing data, comprising:
an annotation database comprising a plurality of annotated k-mers generated from one or more nucleic acid sequences, wherein the plurality of annotated k-mers are annotated with annotation information about the one or more nucleic acid sequences from which the annotated k-mers are generated; and
a processor comprising:
a k-mer extractor configured to extract a plurality of k-mers from a plurality of reads of a nucleic acid sequence;
a k-mer analyzer configured to: (i) identify, using the plurality of extracted k-mers, one or more of the plurality of annotated k-mers in the annotation database found in the plurality of reads; (ii) gather, based on the identified annotated k-mers found in the plurality of reads, annotation information about the plurality of reads; and (iii) determine, based on the gathered annotation information, a quality control metric for at least some of the plurality of reads.

10. The system of claim 9, further comprising an annotator configured to: (i) generate the plurality of annotated k-mers from the one or more nucleic acid sequences; (ii) store the generated plurality of k-mers in the annotation database; and (iii) annotate one or more of the stored plurality of k-mers with annotation information.

11. The system of claim 9, wherein the processor is configured to respond to the determined quality control metric.

12. The system of claim 9, further comprising a user interface configured to provide the determined quality control metric to a user.

13. The system of claim 9, wherein the annotation information comprises information about a location of the annotated k-mer within the nucleic acid sequence, and/or about a characteristic of the nucleic acid sequence from which the annotated k-mer was generated.

14. The system of claim 9, wherein the quality control metric is determined prior to alignment of the received reads to a genomic sequence.

15. The system of claim 9, wherein the k-mer extractor is configured to extract k-mers from the plurality of reads using a sliding window method.
